(19) United States
(12) Patent Application Publication
(10) Pub. No.: US 2005/0281462 A1
(43) Pub. Date: Dec. 22, 2005

(54) System and method of automated processing of multiple microarray images

(76) Inventors: Jayati Ghosh, San Jose, CA (US); Charles D. Troup, Livermore, CA (US); Xiangyang Zhou, Mountain View, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/869,343

(22) Filed: Jun. 16, 2004

(51) Int. Cl. C12Q 1/68

(52) U.S. Cl. 382/173

(57) ABSTRACT

Methods, systems and computer readable media for automatically separating multiple microarray images provided as a single combined image of the multiple microarray images. Methods, systems and computer readable media are provided for providing at least one image containing multiple microarray images thereon, automatically locating the features in the microarray images, automatically determining the boundaries of each microarray image based on the locations of the features, and automatically cropping the image containing multiple microarray images to form a group of single images, each containing only one microarray image cropped from the image containing multiple microarray images. Methods, systems and computer readable media are provided for evaluating separation locations of multiple microarray images in a single combined image of the multiple microarray images, and separating the multiple microarray images along the separation locations, wherein the images are represented in a two-dimensional array.

### BACKGROUND OF THE INVENTION

As microarray technology progresses and becomes more sophisticated, the instruments and methodologies for depositing features of microarrays as well as experiment to interpret results of such experiments with regards to the features enable greater and greater numbers of features to be deposited per unit area of a slide. Precision and resolution of such instruments and methodologies have advanced to the point where multiple arrays are now commonly deposited on a single slide or substrate. However, when working with and interpreting results achieved from experiments performed on such a slide, often referred to as a “multi-pack slide”, users have, to now, needed to first manually separate each microarray image using a software tool, e.g., Agilent Feature Extraction Software (Agilent Technologies, Inc., Palo Alto, Calif.), and organize the images from each microarray contained on a multi-pack slide, from which the user wished to analyze.

Manual separation and organization is tedious and time consuming, and requires manual cropping of each array from the multi-array image resultant from processing the multi-pack slide. The cropped images must then be orderly named, usually with a suffix added to the original name of the multi-pack, to maintain organization and proper reference, and re-saved into the user's database. The re-naming is important to identification of the respective positions of the arrays in the original multi-pack image. The cropped microarray images must be uniquely named for identification purposes for later analysis of the data from each array. Not only is proper naming tedious, but it also increases the opportunity for error, as the user may inadvertently misnumber or misname one or more arrays so as to confuse the order of the experiments as they existed on the multi-pack slide layout.

Hence there is a need to help the processing of multi-pack images to speed up time to processing, relieve users of tedious tasks, and to reduce a source of error associated with research and analysis based on microarray technology.

### SUMMARY OF THE INVENTION

The present invention includes methods, systems and computer readable media. Some embodiments provide for automatically separating multiple microarray images provided as a single combined image of the multiple microarray images. Methods, systems and computer readable media are provided for providing at least one image containing multiple microarray images thereon, each microarray image comprising a plurality of features; automatically locating the features in the microarray images; automatically determining the boundaries of each microarray image based on the locations of the features; and automatically cropping the image containing multiple microarray images to form a group of single images, each containing only one microarray image cropped from the image containing multiple microarray images.

Methods, systems and computer readable media are provided for evaluating separation locations of multiple microarray images in a single combined image of the multiple microarray images, and separating the multiple microarray images along the separation locations, wherein the images are represented in a two-dimensional array.

The present invention further covers forwarding a result obtained from any and all of the methods and techniques described herein, to a remote location; transmitting data representing a result obtained from any and all of the methods and techniques described herein, to a remote location; and/or receiving a result obtained from any and all of the methods and techniques described herein, from a remote location

These and other advantages and features of the invention will become apparent to those persons skilled in the art upon reading the details of the methods, systems and computer readable media as more fully described below.

### BRIEF DESCRIPTION OF THE DRAWINGS

FIG. 1 is a representation of an example of a displayed image resultant from scanning a slide containing multiple microarrays thereon.

FIG. 2 is a flow chart 200 illustrating steps that may be taken in conducting processing according to the present invention.

FIG. 3 shows an example of a user interface that may be displayed to a user of the system, for interactive inputs by a user in conducting processing according to the present invention.

FIG. 4 is a representation of a display of a single microarray image after cropping according to the present invention.

FIG. 5A shows a schematic representation of a slide containing an overall array made up by two microarrays.

FIG. 5B schematically shows a vector resultant from summing the pixels of the slide of FIG. 5A in the X direction.

FIG. 5C schematically shows a vector resultant from summing the pixels of the slide of FIG. 5A in the Y direction.

FIG. 6A shows actual data constructed from the projection of data on a microarray slide in the Y direction.

FIG. 6B shows the data of FIG. 6A, after further processing.

FIG. 7 illustrates the fitting of a Gaussian curve within an identified peak.

FIG. 8A schematically shows an alignment of peaks and the spacings therebetween which are used by the present invention to determine peak spacing for the overall array.

FIG. 8B schematically shows an alignment of peaks and the use of the determined peak spacing to determine group spacing.

FIG. 9 shows a simplified, exaggerated example of an overall array having a first microarray which has been deposited on the slide such that it is rotated with respect to the X and Y axes of the slide and overall array, and a second microarray which is well aligned with the axes.

FIGS. 10A, 10B and 10C illustrate an example of the use of a window function to reduce the sizes of the peaks identified as representative of features.

FIGS. 10D and 10E compare a peak as filtered by the window function, with a peak which has not been so filtered.

FIG. 11 schematically shows another example of a rotated data pattern on a slide, wherein both microarrays of the overall array shown are rotated with respect to the axes of the slide.

FIG. 12A schematically shows a projection, as it appears prior to baseline filtering, in comparison with a projection plot having a perfect baseline.

FIG. 12B schematically shows the unfiltered projection plot of FIG. 12A, as well as plots generated during baseline filtering superimposed thereon.

FIG. 13A schematically shows a plot of peak groupings in which one of the groups presents with a peak missing.

FIG. 13B schematically shows a plot of peak groupings in which an anomalous peak makes two groups appear as one large grouping.

FIG. 13C shows the plot of FIG. 13B after processing, wherein the large grouping has been broken down into two proper sized groupings.

FIG. 14 is a block diagram illustrating an example of a generic computer system which may be used in implementing the present invention.

### DETAILED DESCRIPTION OF THE INVENTION

Before the present systems, methods and computer readable media are described, it is to be understood that this invention is not limited to particular hardware, software, methods, method steps or algorithms described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a row” includes a plurality of such rows and reference to “the image” includes reference to one or more images and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

In the present application, unless a contrary intention appears, the following terms refer to the indicated characteristics.

“Projecting” or “projection” of a two dimensional image onto a one dimensional line refers to adding all the values or selected values within the same row or column index of a matrix to yield a one-dimensional dataset with the same length as one of the dimensions of the matrix or the length of the matrix taking into account the selected values, i.e. number of rows or number of columns. “Simple projection” may be performed without any image rotation or additional image processing. Simple projection works well when the features are well aligned and well-formed, as a line of such features along the projection direction will form a sharp peak on the projection. Because simple projection is linear, its result is dominated by large intensity signals on the image being projected, regardless of whether the large intensity signals are caused by features or spurious pixels (e.g., scratches, drying traces, gasket traces, etc.) Further, the projection of a doughnut shaped feature will appear as a double peak, separated by the inner diameter (i.e., “doughnut hole”) of the doughnut shaped feature. If the features are poorly separated, or if the rows and columns of the grid are not exactly aligned along the rows and columns of the image, the projection will appear blurred, as the expected peaks will bleed into those formed by neighboring features.

“Non-linear projecting” or “non-linear projection” is the same as “projecting” or “projection”, except that preprocessing is done before the projection. Such preprocessing may include computing local minima or maxima, taking the logarithm of the local minima or maxima, computing projections along rotated axes as needed, and/or dithering sums over the one-dimensional data set.

“Projecting based on orthogonal projection” includes any of the above projection techniques wherein the row or columns that are summed are only those that are near a local maximum in the other dimension (row or column). Typically, only the middle half (or some other predefined central portion) of those maxima are considered.

“Gauss filtering”, “Gaussian Filtering” or “Gaussian Integration” involves a classical application of a correlation with a Gauss kernel.

“Large trace removing” involves addressing and filtering artifacts caused by gasket traces, drying traces, or other large artifacts on the slide/chip which, if not treated, tend to show up as very bright, large areas when viewed.

“Zero rank filtering” involves removing the local baseline under the one-dimensional data after it has been projected.

“Peak picking” a one-dimensional dataset involves finding all the local maxima, and further processing the local maxima to determine which are features to be kept for data interpretation.

“Spacing estimation” involves the computation of the most frequent distance between adjacent peak centers.

“Peak height selection” involves statistical processing to weed out peaks that are created by image artifacts.

“Block finding” involves forming groups of peaks from the total population, based on sets which are generally equally spaced according to a given or measured spacing. Blocks are intended to define microarrays within an overall array.

“Block size computing” involves computing to choose the block size that involves the maximum number of peaks, after peak picking has been performed.

“Block fixing” attempts to force a given block size on blocks that are either smaller or larger than the computed block size.

A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

An “array”, “microarray” or “bioarray” unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably. A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice).

A “microarray” is a subset of an overall array as presented on a multipack slide. Typically, a number of microarrays are laid out on a single slide and are separated by a greater spacing than the spacing that separates features or spots or dots. The terms “subarray” and “array” or “microarray” may be used interchangeably, depending upon the context. For example, in the situation where multiple arrays are laid out on a single slide, each array may be considered a subarray of the entirety of the layout, which could be considered an array made up of the subarrays, wherein each subarray may be an independent microarray, such as referred to in the present description, and wherein the array formed as a composite of such subarrays may be referred to as the “overall array”.

Any given substrate (e.g., slide) may carry one, two or more (e.g., many now have eight) arrays disposed on a front surface of the substrate. Depending upon the use, any or all of the arrays may be the same or different from one another and each may contain multiple spots or features. A typical array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range from about 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features).

Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used,. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible; for example, some manufacturers are currently working on flexible substrates), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods.

Following receipt by a user of an array made by an array manufacturer, it will typically be exposed to a sample (for example, a fluorescently labeled polynucleotide or protein containing sample) and the array then read. Reading of the array may be accomplished by illuminating the array and reading the location and intensity of resulting fluorescence at multiple regions on each feature of the array,. For example, a scanner may be used for this purpose which is similar to the AGILENT MICROARRAY SCANNER manufactured by Agilent Technologies, Palo Alto, Calif. Other suitable apparatus and methods are described in U.S. Pat. Nos. 6,406,849, 6,371,370, and U.S. patent applications: Ser. No. 10/087447 “Reading Dry Chemical Arrays Through The Substrate” by Corson et al., and Ser. No. 09/846125 “Reading Multi-Featured Arrays” by Dorsel et al. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,251,685, U.S. Pat. No. 6,221,583 and elsewhere). A result obtained from the reading followed by a method of the present invention may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

Whole genome screening using current high-density oligonucleotide microarrays has yielded valuable information to help researchers identify key biomarkers or pathways of interest. Lower density microarrays capable of screening a few hundred to a few thousand genes of interest can be used to perform detailed screening of specific disease states or evaluate the toxicity of certain drugs against target organs, for example. Further, hybridization platforms exist (e.g., available from Agilent Technologies Inc., Palo Alto, Calif.) that accommodate lower sample volumes and permit parallel processing and screening of multiple microarrays on a single slide. Thus, multiple microarrays, for example, up to eight microarrays, may be processed on a single 1″×3″ slide.

To analyze results obtained from microarray experiments where multiple microarrays are provided on a single slide, the researcher or analyst first needs to have the multiple images which are produced from the multiple microarrays located on the single slide, separated or cropped, so that the researcher or analyst can work with data from a single microarray (i.e., single image) at a time, since generally the researcher or analyst is interested in observing the data from only one microarray at a time. Even when later comparisons are to be made between microarrays, the initial analysis is generally done with regard to each individual microarray prior to such comparisons. Further, current available processing software programs analyze only one microarray at a time.

Current methods for such pre-processing require manual cropping and naming or cataloguing of the cropped images, as noted above. Embodiments of the present invention eliminate the need for such manual tasks, thereby reducing the chances for erroneously naming or organizing the cropped images, and simplifying pre-processing by automating it.

FIG. 1 is a representation of an example of a displayed image 100 resultant from scanning a slide containing multiple microarrays thereon. In this case there were eight microarrays on the slide that was scanned (“eight-pack microarray”). As can be observed from each microarray image 110, each microarray has been laid out in an orderly fashion on the slide, in rows and columns forming an array. The microarrays (represented by images 110) may all have the same corresponding probes with the same experiments run on them, may all have the same corresponding probes with different experiments run on respective microarrays, or may have different designs. Typically, the microarrays will be separated by margins 112,114. After inputting one or more such images from multi-pack slides into the present system, the present system performs automated processing necessary to “split” the multiple images into a series of individual images each of a microarray 110 and independently names or titles each such image.

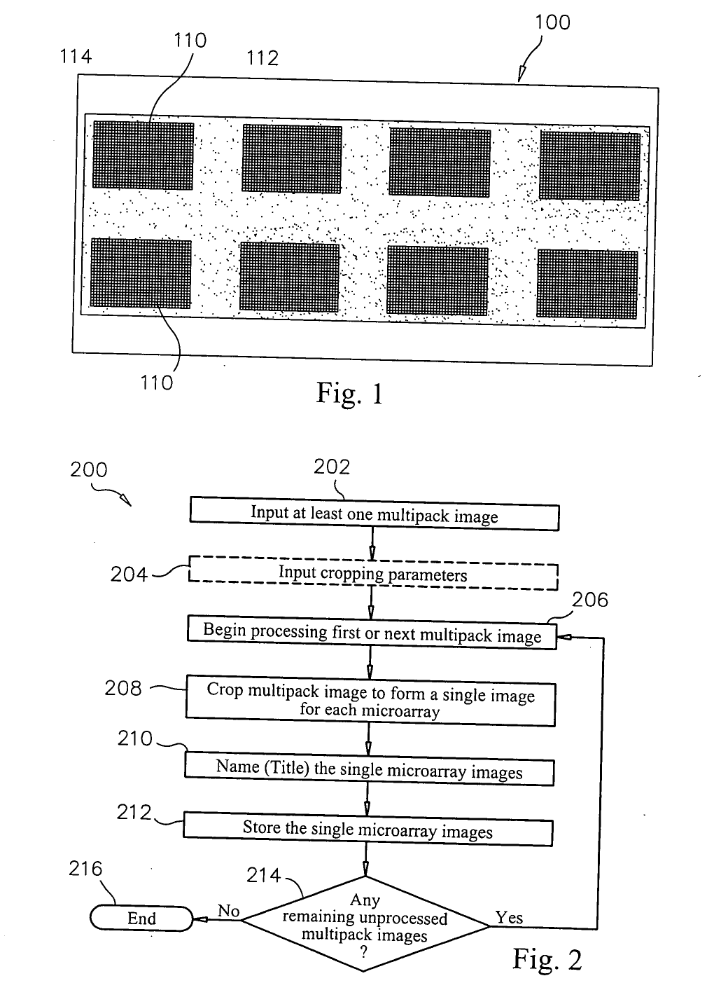

FIG. 2 is a flow chart 200 illustrating steps that may be taken in conducting the processing referred to above. After inputting at least one multi-pack image to the system at step 202, the user may optionally specify cropping parameters, such as layout characteristics of the multipack array at step 204. However, this input is optional, as the system will automatically determine the layout characteristics of each multipack array during processing, as will be discussed below. Accordingly, the system is capable of batch processing multipack array images, regardless of whether each image contains the same number of arrays and same layout or not.

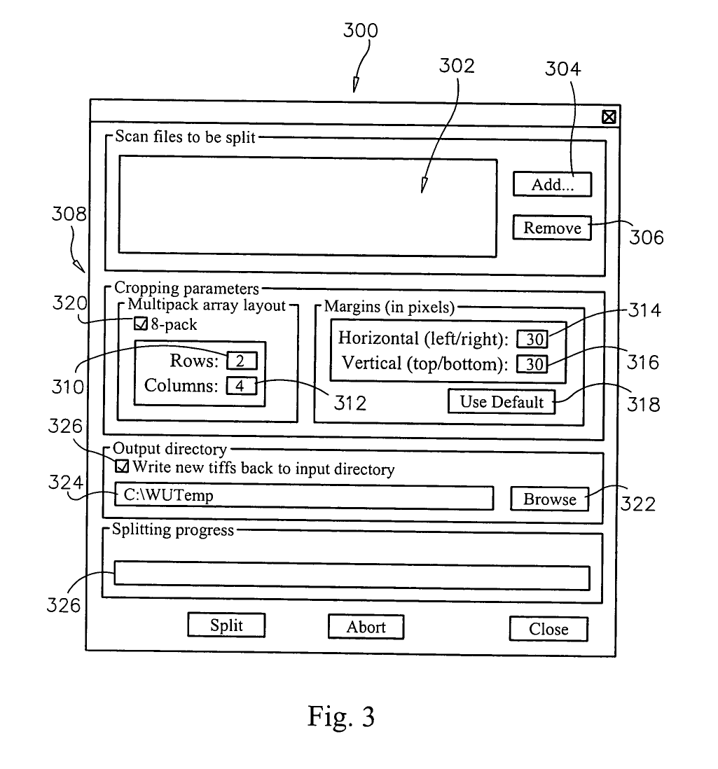

FIG. 3 shows an example of a user interface 300 that may be displayed to a user of the system. Input box 302 is where the image files to be processed are displayed and inputted to the system at step 202. The user may add files to be processed by selecting from a database of files using the add button 304, or conversely, may remove files from input box 302 using the remove button 306, if it is decided not to process one or more selected image files at that time.

Provisions for optional input of cropping parameters (step 204) are made at portion 308 of user interface 308, where the user may input all, a portion of, or none of the parameters of the multipack array or multipack arrays to be processed, depending on the conditions of the particular run. For example, if the user is processing a single multipack array image 100 wherein the specifications as to row and column layout are known, as well as marginal specifications, then the user may input this information. The number of rows of microarrays in the layout of the multipack array 100 may be specified through input box 310 and the number of columns of microarrays in the layout of the multipack array 100 may be specified through input box 312. Horizontal and vertical margins are defined as number of pixels from the outermost columns and rows to the respective edges of the cropped image, and these margins may be inputted at input boxes 314 and 316, respectively. A standard, default setting may be stored for parameters of margins, rows and columns of the most common multipack images processed by the user. In this case, the user may simply select the default button 318 to apply the default settings. Additional “template” or default settings may be provided for, such as the check box 320 that is provided to automatically set the rows, columns and margin parameters for the most common 8-pack images for this example.

Another example for setting the above-described parameters is where a user is batch processing a plurality of multipack array images 100 that are all laid out with the same parameters. However, in cases where the user is processing one or more multipack array images for which one or more of the parameters is unknown, such parameters need not be inputted, as the system can determine them during processing. The user may specify the margins to be established after cropping, or, if not specified, the system will default to establish margins of predetermined default size (e.g., 30 pixels horizontal margin and 30 pixels vertical margin, or other predefined settings). By specifying the number of rows and columns in a microarray via the user interface, this increases the probabilities of accurately locating and cropping the microarrays, particularly when microarrays having skewed features/probes or other errors are present, such that the projections are not very well defined. Similarly, even if all parameters are known, but the user is processing a batch of multipack array images of which at least one has different parameters then the other, then these parameters may be omitted, allowing the system to automatically determine parameters for each multipack array image.

User interface 300 further permits the user to specify where the user wants the output files (i.e., single microarray image files) to be stored upon completion of processing. Through the use of browse button 322, the user may browse a directory and select a location displayed in the browse window 324. Alternatively, the user may select check box 326 to automatically store the output files back into the same directory from which the multipack array images were inputted. It should also be noted here, that the image files that are processed by the system are typically TIFF files, as this is a common format for formatting microarray images. However, the present invention is not limited to TIFF files, as other file formats may be processed similarly, e.g., BMP, JPG, GIF, etc. Processing progress may be displayed as a bar graph in window 326.

After inputting the multipack image(s) to be processed, and optionally inputting cropping parameters, the system begins processing the first inputted multipack image to perform the cropping operations at step 206. The system preferably uses a projection-based algorithm to locate the features on the microarrays on the multipack image. Although it is possible to consider using a fast-Fourier transform (FFT)-based algorithm in performing this stage of processing, projection-based processing provides advantages as discussed hereafter. A goal of this stage of processing is to calculate the precise location of each array in the multipack image. Though an underlying assumption is that all microarrays are placed evenly and periodically on the slide from which the multipack image is generated, this may not always be the case due to potential errors induced in manufacturing the slide. The projection-based algorithm accounts for inconsistencies in the placement of microarray slides and adjusts the positions of individual arrays in both x- and y-directions along the multipack image. When using an FFT-based algorithm, however, the calculated interarray spacings are exactly the same for all the arrays on a slide. When using an FFT-based algorithm, the array spacing primarily relies on the peak frequency of the initial projection data, and will be less accurate if there is no periodicity information in a given direction, such as, for example, when the layout includes only one row and multiple columns of arrays. Also, since the precise locations of the microarrays may not be determined by use of an FFT algorithm, since the same spacings are used between each microarray, which may not realistically describe the actual layout, cropping of images may not be as precise and larger margins may need to be left around the images to account for tolerances. Much closer cropping can be confidently performed when based upon a projection-based algorithm.

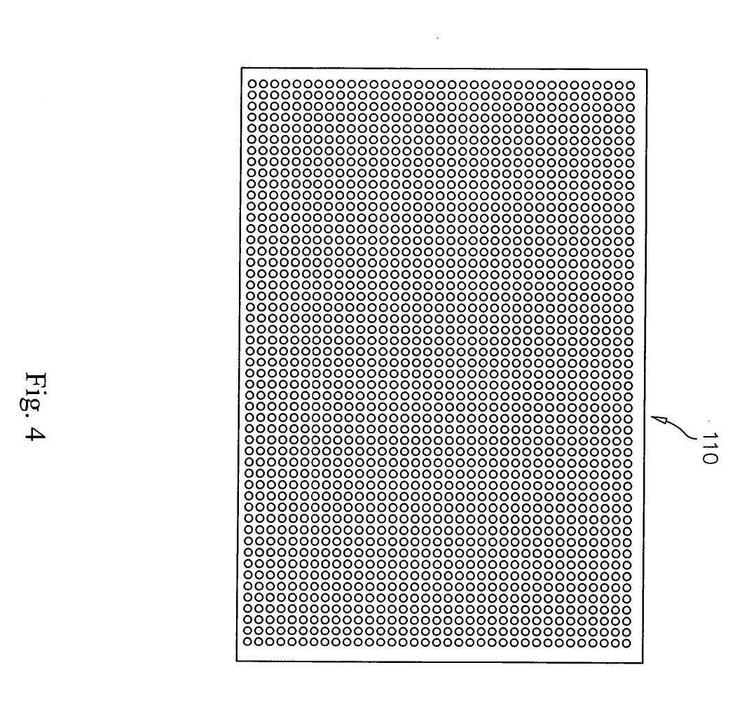

Once the locations of the microarrays 110 have been determined on the multipack image 100, the system crops the images at step 208, thereby creating a single image for each microarray 110. A display of a single microarray image 110 after cropping is represented in FIG. 4. The images of single microarrays 110 are named by the system (step 210) in a way that maps them to their original locations on the multipack image from which they were cropped. For example, for an eight-pack multipack image named “multipackexp” the cropped image containing the leftmost and topmost microarray in the multipack image may be named “multipackexp_{—}1_{—}1”. The cropped image containing the microarray immediately to the right of “multipackexp_{—}1_{—}1” but still in the same row, may be named “multipackexp_{—}1_{—}2”, the cropped image containing the microarray immediately below “multipackexp_{—}1_{—}1” but still in the same column, may be named “multipackexp_{—}2_{—}1”, etc., where the suffix “-a-b” refers to the row and column where that microarray was located on the slide (a being the row number and b being the column number). Of course, other naming conventions may be developed that would also accurately locate the origins of each microarray on a slide.

The cropped image files are then stored at step 212, into the directory designated by the user or a default directory. The directory may be the input directory as a default, may be chosen as the input directory by the user, or may be some other directory chosen by the user, or some other default directory. After storage of the current output files (single microarray images), the system then checks to see if there are any remaining input files (multipack images) which have not yet been processed (step 214). If there are no remaining input files to be processed, processing ends (step 216). If this is a batch process and at least one input file remains to be processed, processing returns to step 206 to begin processing the next input file in the manner that was described previously. Steps 206, 208, 210 and 212 are then iterated for each remaining input file until all input files have been processed, at which time processing ends.

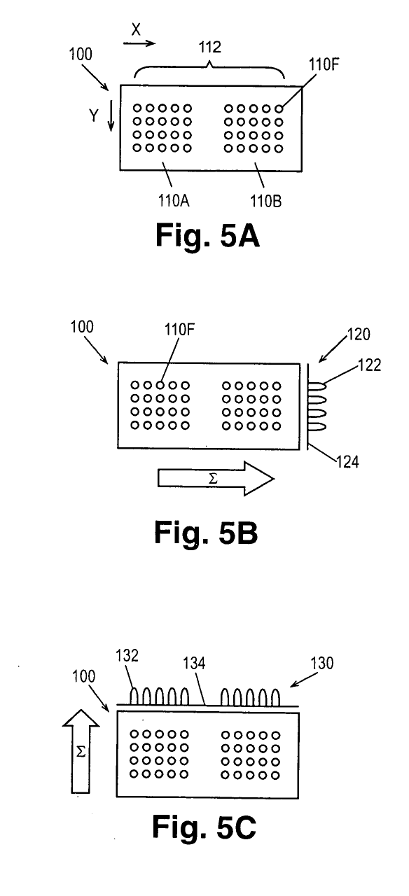

Referring now to FIG. 5A, a further description of a projection-based algorithm that may be used for processing as described herein is discussed. A more complete description of such projection-based algorithm may be found in co-pending, commonly assigned application Ser. No. 10/449,175 filed May 30, 2003 and titled “Feature Extraction Methods and Systems”. Application Ser. No. 10/449,175 is hereby incorporated herein, in its entirety, by reference thereto. Further information regarding feature extraction techniques can be found in U.S. Pat. No. 6,591,196, which is also incorporated herein, in its entirety, by reference thereto.

A goal, when examining a slide containing multiple microarrays, is to locate the layout of the microarrays contained on the slide (e.g., the image thereof). The approach taken by this process is to locate the “dots”, “spots”or “features” as they are arranged and spaced on the slide, at the same time determining their groupings into separate microarrays. By identifying where the features reside, this makes it further possible to ignore or filter out other information which is not located where the features are.

An initial approach to locating the features on the multipack image aims to locate the centers of the features. To begin with, the slide containing the microarrays is read to sum the rows and sum the columns of the overall array intensity made up by all microarrays on the multipack image to create the projections of the two dimensional image formed by the slide along one dimensional lines. By condensing the data from a two dimensional image to two vectors of one dimensional data, this greatly reduces processing time, since the processing time required to process one dimensional data is the square root of the time for processing the two-dimensional image data.

If the dots or features are thought of as bumps or hills in the intensity domain, the projection process endeavors to look in the plane of these features to determine the skyline or topography of the features. If there are a few missing features here or there, it doesn't matter to the projection, because there are enough present, so that statistically, all of the projections will have about the same height. Also, even if most of the spots are faint, the sum of all those values are going to be significantly higher than the background signal. This provides an additional advantage over two-dimensional processing because of the increased signal to noise ratio produced by summing the features.

By locating the centers of all the features (peaks which are determined to represent features) in one dimension, and the centers of all those peaks in the second dimension, the system identifies the grid (overall array on the multipack image) of data represented as features on the microarrays. By finding centers, this gives the “x” and “y” coordinates for each feature which are then used to identify the location of the overall array.

FIG. 5A shows a schematic representation of a slide 100 containing an “overall array” 112 made up of individual microarrays 110A and 110B. Of course, this schematic representation is greatly simplified, as a typical slide will contain many more features 110F and may contain more microarrays 110, but for purposes of illustration and explanation, the number of features has been shown much smaller and only two microarrays 110 have been shown.

The convention used for the microarrays and overall array on slide 100 in FIG. 5A and throughout this application is that the X-axis runs horizontally, from left to right, along the slide as shown in FIG. 5A, and the Y-axis runs vertically, from top to bottom, along the slide 100. To perform the projections, the sum of all the rows is performed and the sum of all the columns is performed. Thus, the projections in the X and Y directions are as follows:

Projection for X:

$$A ( x ) = {\sum }_{y} f ( x , y ) \qquad ( 1 )$$

where
A(x) is the projection value for a full column of intensity values aligned along a given “x” pixel location; and
f(x,y) is the intensity of the illumination of the pixel at the given x and y coordinates.

Projection for Y:

$$B ( y ) = {\sum }_{x} f ( x , y ) \qquad ( 2 )$$

where
B(y) is the projection value for a full row of intensity values aligned along a given “y” pixel location; and
f(x,y) is the intensity of the illumination of the pixel at the given x and y coordinates.

The result of performing the projections reduces the matrix of intensity values provided by the overall array 112 on slide 100 to two vectors of values. FIG. 5B schematically shows the vector 120 resultant from summing in the X direction, and FIG. 5C schematically shows the vector 130 resultant from summing in the Y direction. The bumps 122 and 132 represent the illumination peaks resultant from summing the high illumination areas where the features are located. The baselines 124, 134 represent spaces on the slide which are not occupied by features l OF. Of course, these schematic representations show idealized final results of processing, which are generally achieved after the further processing to be discussed hereafter. Upon forming the initial projections, however, the baselines 124, 134 are usually irregular and may contain peaks of their own which can be caused by any of the factors described above that cause illumination which is not representative of a feature.

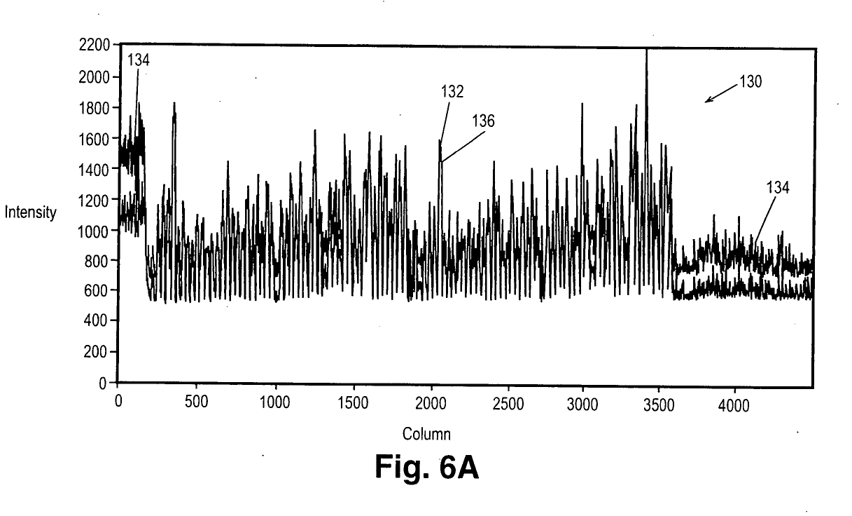

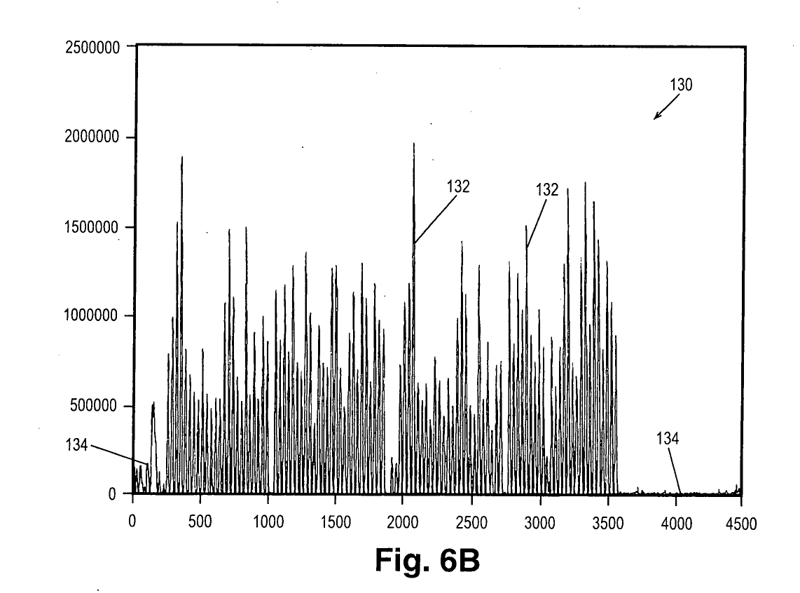

FIG. 6A shows actual data constructed from the projection of data on a slide 100 in the Y direction. It can be seen that the baseline 134 is erratic and shows some illumination, possibly caused by a gasket seal mark left on the slide. After making the projections, the data may be further processed, so that it may be more effective for use in determining the layout of the overall array and/or microarrays and features. FIG. 6B shows the data of FIG. 6A, after further processing. The projections are used to find which are the meaningful peaks which are representative of loci of the features. The center of these peaks is desirable to find since this increases the probability of locating the column or row coordinate. Baseline processing is done to filter the spurious sources of illumination and return the baseline much more closely to a constant line indicating little to no illumination.

A smoothing function may also be applied to the projection to get rid of the higher frequency minor points (“jitters”) 136 which may be superimposed upon the major peaks. The smoothing of the peaks makes it easier to discern the actual peaks that are representative of the features. For example, a correlation with a Gaussian kernel that is a few points wide (typically three to five points wide, using 10 micron pixels or points) yields appropriate smoothing.

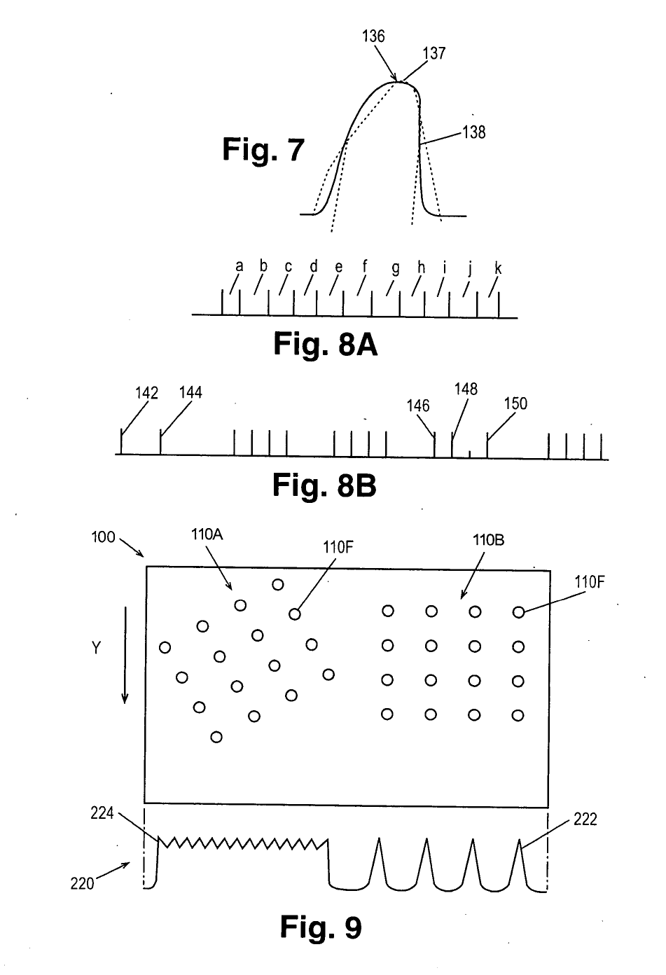

After smoothing the local maxima of the plots are determined. Then, an interval is taken around each local maximum, and a Gaussian curve is fitted in the interval. The center of the Gaussian curve 136, which may be different from the peak maximum 137, is found using a centering algorithm. Typically the centroid algorithm (i.e., where the center of gravity of the peak is computed) gives satisfactory results. Additionally, the area under the curve defined by the peak within the interval is calculated, as well as the peak width (half-width maximum) 138 (see FIG. 7). This reduces projection curves to a list of identified peaks, which is just two lists of numbers. This is important because it further reduces the number of points subjected to subsequent, more complicated processing, thereby reducing the time and cost of locating the microarrays to be cropped. For example, for a 6000×2000 pixel overall array 112, this gives twelve million data points to be processed. Projection processing reduces the number of data points to be processed to around eight thousand (six thousand plus two thousand, plus possibly some artifacts). By finding the peak centers, the number may be reduced to only a few hundred data points to be further processed (e.g., about 200-500 points).

Next the peak shapes are statistically processed in an effort to recognize and filter out the peaks which do not fit the shape of the general population (i.e., filter out the outliers) and which are therefore most likely to be representative of noise caused by artifacts, rather than illumination caused by features. Statistics may be done on the area, as well as width of the peaks, in an effort to filter out the peaks that do not fit the general population (i.e., to identify the outliers, which are most probably noise masquerading as peaks). The median value of the areas under the peaks and the median peak width are calculated, and peaks that have a significant variation from these median values are discarded from the set of peaks to be considered for viewing as features. Peaks that are determined to show a significant variation are those peaks that have an area that is more than a predetermined amount less that the median area (for example, twenty times smaller than the median area) as well as peaks whose width is more than a predetermined amount greater (e.g., at least about 50% greater) than the calculated median width.

It is not practical to attempt to identify peaks having a height that is significantly higher than the general population, because the data may be such that most of the features are very faintly illuminated, with one or a few being very intense. Of course, these very intense features are features which should be considered. Also, there is no need to remove peaks that are too narrow, because such peaks are also usually too small in area, so as to be effectively filtered by the median area filter.

Next the system endeavors to find the spacing between peaks. The spaces between each pair of adjacent peaks are calculated and tabulated, after which, the median difference between adjacent peaks is calculated. The median value is then set to be the feature spacing, i.e. distance between adjacent features in the dimension being considered. Although the median is the preferred measure for determining peak spacing, it is noted that other statistical measurements could be substituted for peak spacing. For example, some other form of “average” calculation could be employed to determine peak spacing, although some approaches may not be as accurate, since the spacing distances between microarrays 110 will generally be calculated along with the interfeature distances within microarrays 110. Using the median measure in FIG. 8A, as an example, if the spacing between the peaks shown in FIG. 8A is a, b, c, d, e, f, g, h, i, j and k, respectively, and if these spacing distances, arranged from smallest to largest spacing are: a, k, i, h, c, d, g, h, e, b, f; then the median spacing is d.

The peak spacing may be further used to determine group spacing, e.g., distance between microarrays 110. In the example shown in FIG. 8B, the groups show a general tendency to have four peaks per grouping. Although only four peaks per group are shown, this is for simplicity of explanation, as in practice, there are usually many more features per microarray 110 in either the X- or Y-direction, so spacings between the microarrays don't effect the median all that much, when finding the median spacing. The system then analyzes the peak spacings to define the microarrays 110. Spacings that fall within a predefined range about the median spacing (e.g., median spacing ± about 20%) are considered to be spacings between adjacent features 110F within a single microarray 110. Peaks are clustered in groups according to these spacings. Then the groups are sorted by size (i.e., number of peaks per group), and the size which explains the majority of the groups (i.e., the number of those groups times the number of peaks in that group size) is deemed to be the microarray size. Then the groups are sorted by position, and the average of the distances between adjacent groups of the determined group size is assigned as the microarray spacing distance.

By the foregoing techniques, the system determines that the peaks shown in FIG. 8B include groupings of 1, 1, 4, 4, 2, 1 and 4 peaks, respectively. The system then chooses the grouping number that characterizes the majority of the peaks in the dataset to determine the number of peaks in a group or microarray 110. For example, in FIG. 8B, three peaks are characterized as groupings of one peak, two peaks are characterized as groupings of two peaks, and twelve peaks are characterized as groupings of four peaks. From this the system concludes that the microarrays 110 are characterized by groupings of four peaks each. Therefore, peaks 142 and 144 are considered to belong to a first microarray 110, and peaks 146, 148 and 150 are considered to belong to a fourth microarray 110 in this example.

All of the preceding procedures may then be repeated in the second dimension to determine peaks and spacing between microarrays 110 in the other dimension. For example, if projection, etc. is performed in the X-direction first, then the procedures are repeated in the Y-direction, or vice versa.

The projections are easiest to calculate when all of the features are well-formed and consistent, and the overall array 112 and microarrays 110 forming the overall array 112 are all aligned with each other as well as with the slide. However, in reality, many discrepancies from this ideal layout occur. For example, the entire array 112 may be rotated with respect to the X and/or Y axes. Additionally or alternatively, one or more microarrays 110 may be rotated with respect to the other microarrays 110 in the array 112. Also, rows and/or columns of one or more microarray 110 may be misaligned with rows and/or columns of adjacent microarrays 110. FIG. 9 shows a simplified, exaggerated example in which microarray 110A of multipack image 100 has been deposited on the slide such that it is rotated with respect to the X and Y axes of the slide. In comparison, microarray 110B is well aligned with the axes. The projection plot 220, resultant from taking the projections along the Y axis, shows well formed peaks 222 with respect to microarray 110B, since the features 110F are aligned with the Y axis. However, the projection of microarray 110A shows numerous peaks 224, the spacing between which is not easily discerned. This is due to the misalignment of the columns of features 110F of microarray 110A with respect to the Y axis. The resultant projection is not useful for determining spacing between features.

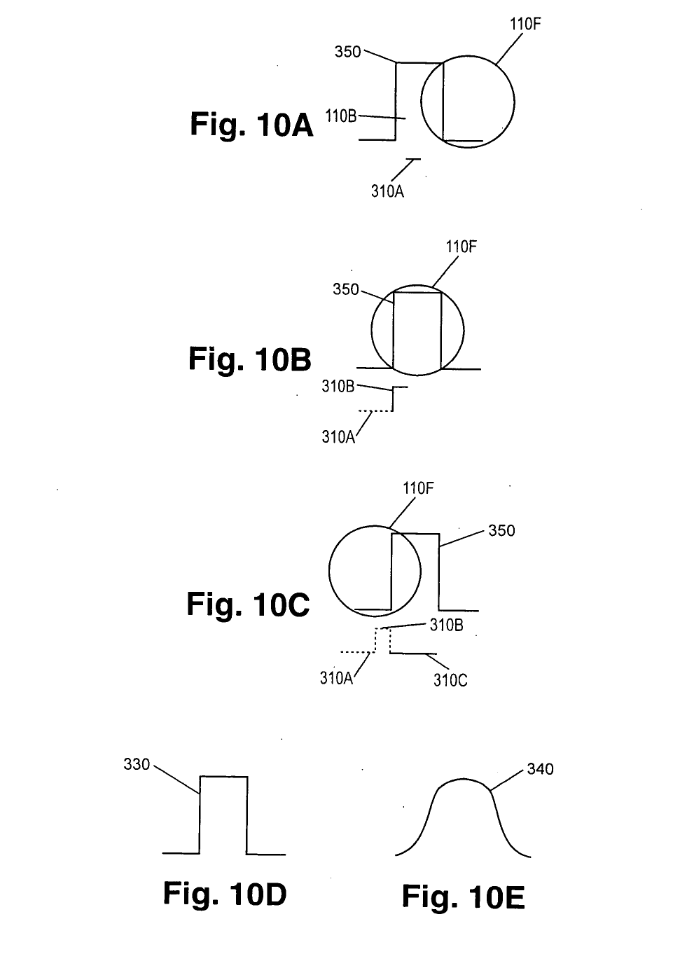

way of dealing with this problem is to reduce the size of the features 110F. To do so, the system filters the reading of the features during the projection process, by recording the minimum value within a window at each pixel position as it passes over a feature 110F. The window function has a width that is smaller than the width that the features 110F are usually produced to have. For example, features 110F may be formed to have a width of about 15 to 30 pixels, and the window used may have a width of about 7 pixels. FIGS. 10A-10C show representative values taken as the window 350 moves across a feature 110F to record the minimum intensity levels during processing. At FIG. 10A, because a portion of window 350 is not yet sensing feature 100F, the minimum level will be somewhere near representative of the baseline, as indicated by plot 310 A, since window 350 is still reading background 110B on the slide, and the background is generally not illuminated.

FIG. 10B shows a position of window 350 where everything that it is sensing is within the confines of feature 110F. Accordingly, the minimum illumination value sensed at this position is going to be relatively high, since the entire area of the window is illuminated. Hence, the plot 310B reflects this high reading. FIG. 10C, like FIG. 10A, shows a position of window 350 where the entire window is not reading feature 110F. Hence, the minimum illumination value will again be near baseline, as indicated by plot 310C. Although only three representative positions of window 350 are shown with respect to feature 110F, this process may record a minimum illumination value for many more loci, and may record minimum values on a pixel by pixel basis. Also, window 350 may have multiple positions where feature 110F is being read in the entire area of the window, since, as noted above, window 350 may be much narrower than the width of feature 110F.

This filtering process results in a much narrower, more well-defined peak representative of each feature 110F read. FIGS. 10D and 10E illustrate the difference between a filtered peak 330 and a non-filtered peak 340. By narrowing the peaks, this results in much clearer, more well-defined peak spacing, which is easier to process for determining the feature spacing and microarray layouts, and also prevents overlap of one column or row of features with an adjacent column or row, up to a certain extent of rotation of a microarray. This filtering is also useful to filter out noise or spurious signals where the anomaly is less than the width of the window used.

The present invention need not determine an accurate reading of the intensity value of a feature 110F, or even of the particular shape of each feature. Rather, the process is performed for targeting the locations of the features 110F. Therefore, the logarithm of the intensity values are used during processing to curb the overall effect of the large intensity values (privilege or weight the general population of intensity values versus those which are abnormally high). This may be useful to downgrade the importance of anomalous sources of illumination which may present with higher intensity than the features.

A further refinement of projection processing may be performed to increase the signal to noise ratio of the resulting projections. This refinement involves computing projections of all of the pixels only for the first projection performed, whether it be in the Y direction or the X direction. After obtaining the first projection plot in the manner described above, the system identifies only the location where peaks representing features 110F are suspected. The locations of the peak maximums are the result of the peak-picking algorithm that has been performed in the current dimension of processing.

Once the suspected peak locations are identified in the first dimension, only those pixel lines (columns or rows) corresponding to the identified peaks (and a predetermined distance on either side of each peak (e.g., for typical feature sizes and using 10 microns pixels, about 3-8 pixels on each side, preferably about 4-6 pixels on each side) are processed for a projection in the other (X or Y direction). This not only reduces processing time, but eliminates a lot of the background noise in between the rows or columns of the features 110F which need not be processed. For example, if the first projection is in the X direction, then the identification of peaks from this first projection narrows down the rows of pixels which are to be considered. Then, when subsequently performing the projection in the Y direction, only those column values which lie in the identified row positions are considered during the projection. The same process can be applied if the first projection is done in the Y direction, wherein, when doing the subsequent X projection, only those row values which lie in the identified column positions would be considered.

Returning to the first example, after projection in the X direction, selection of peaks, and then projecting in the Y direction using only selected row positions, the projection in the Y direction can be processed as described above, to find peak centers of the features 110F (e.g., using window 350), to determine the spacing between features 110F and to determine the layout of the microarrays. Then another projection is done in the X direction using only the identified peak locations from the Y-projection to limit the column positions of the row pixels that are projected.

The system may further apply the process steps described above in order to determine the rotation (if any) of one or more microarrays in the overall array. FIG. 7 shows another example of a rotated data pattern on grid or slide 400, wherein only two microarrays 410A and 410B are shown on the slide 400 for sake of simplicity. In order to find the rotation, the same methods are used to try to locate the positions of the features 110F. However, the process is performed separately on two different portions of the data, to determine differential data patterns (i.e., locations of features 110F). In the example shown, projections and further processing are performed on the first portion (which could be a quarter or other predetermined fraction of the data set) 410A and last portion (which is generally equal to the portion against which it is to be compared, e.g., a quarter or other predetermined fraction) 410B.

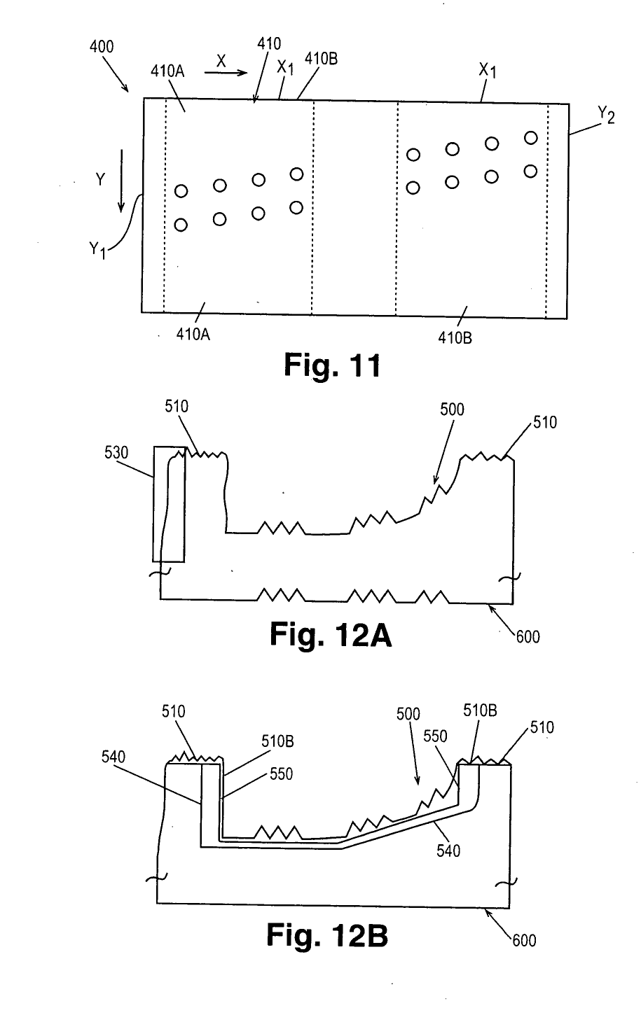

The location patterns of features 110F of each of these portions is then compared to determine the offset, in both the X and Y directions. Using the offset values, the degree of rotation can be readily calculated. For example, in FIG. 11, the processing determines that portion 410A contains a 2×4 microarray with an origin of the microarray at Y position y₁. Similarly, the processing determines that portion 410B contains a 2×4 microarray with an origin Y-position at Y position y₂. The average X-position position of microarray 410A is determined from the processing to be x₁ and the average X-position microarray 410B is determined to be x₂. From these values, the angle of rotation may be determined by calculating a tangent, i.e., (y₂−y₁)/(x₂−x₁). The rotation is then subtracted out. Until the rotation is computed, the projections are parallel to the X and Y axes. By computing the rotations, the projections can be performed along a rotated frame, i.e., along axes that are at an angle to the X and Y axes, by an angle derived from the degree of rotation in each dimension. The first projection is generally done parallel to the shorter dimension of the scan (grid), onto the larger dimension. Only a portion of the short dimension is projected (e.g., a quarter of it, as noted above) to minimize the effect of the rotation. Once the first partial projection is performed, the rotation is computed by looking at the offset between both ends in the second dimension.

After subtracting out the rotations determined along the first dimension, processing of portions is repeated in the other dimension, in the same manner as described above. The rotational results in this dimension, combined with the rotational results in the first dimension (described in detail above) determine the skew of the pattern. The skew is the difference between the rotation with respect to the X and Y axes. Put another way, the skew is the rotation left in the second dimension after the image has been rotated along the value found in the first dimension. A skew pattern is caused by rotation with respect to both axes and is probably most easily described as a pattern that looks like a parallelogram that does not have right angles. By subtracting out the rotation with respect to both axes, the feature centers can be accurately located over the whole grid, i.e. overall array.

Baseline Processing

An example of baseline processing involves filtering sources of illumination which have a period (width) that is substantially greater (e.g., twice, or some other predetermined multiple) than the width (or expected width) of the peak spacing (i.e., distance between centers of the features 110F. The predetermined multiple may vary depending upon how much information is known about the overall array prior to processing. For example, if no information is known, the predetermined multiple may be about twice the expected peak spacing. If the peak spacing has already been specified prior to processing, the predetermined multiple may be about 1.5 times the peak spacing, or even equal to the peak spacing. In one example where no information is known, a window spacing of 31 points (pixels) is used (assuming pixel size of 10 microns).

This baseline filtering process may employ a window function that operates conceptually similarly to the window function used for reducing the peak size, as discussed above with regard to FIGS. 10A-10C. Referring now to FIG. 12A, a projection 500 is schematically shown, prior to baseline filtering. Beneath projection 500, a filtered projection 600 is shown which has the baseline reduced to somewhere around a zero background level. The baseline of projection 500 in this example includes very high intensity blocks 510 such as which can be caused by a gasket seal mark left on the slide. The entire baseline is biased substantially above the zero background level, and portion 520 has a baseline with an increasing slope.

As noted above, the window 530 for the window function is selected to be no smaller than the peak spacing or expected peak spacing, and is generally about 1 to 2.5 times the peak (or expected peak) spacing. As the window 530 is passed over the projection 500, the minimum value observed in the window is obtained for each progressive position of the window 530 over the projection 500. Window 530 may be advanced by as little as one pixel between each position for which a minimum value is obtained, or a larger incremental movement may be employed for faster processing. However, by performing the projections as noted, this typically reduces the number of points to consider to somewhere in the neighborhood of about 6000. With this reduction, it is possible to advance one pixel at a time and still complete the processing very quickly, as the reduced information for the entire grid (array) can be loaded into a processor cache. FIG. 12B shows a plot 540 constructed from the minimum values obtained as window 530 passes over the entire projection plot 500. If plot 540 is subtracted from plot 500 the result is a projection plot with a baseline much closer to the zero luminance line that is desired, with the exception of the block portions 510B that are not captured by the first window filtering process.

To remove the remnant block portions 510B, a reverse transformation is employed, wherein maximum values of plot 540 are obtained using the same window function. The plot 550 resulting from the maximum value filtering step is also shown in FIG. 12B. Curve 550 approximates the projection curve much more closely than curve 540, as can be seen. By subtracting curve 550 from curve 500 a very effective filtering of the baseline is accomplished, approximating curve 600.

Peak Width Measurement and Gaussian Fit

When finding the peak centers as described above with regard to FIG. 7, a rather conservative window (e.g., a relatively narrow window on the order of about 11 pixels (center plus five pixels on each side), although a smaller window as small as 7 pixels may be used) may be used to find the peak centers. While this approach is useful for finding peak maxima and peak centers, it may not be very accurate for computing peak widths, as the relatively narrow window will not capture enough of the peak width of a relatively wide peak, since the window is not wide enough to do this.

Accordingly, once the peak spacing has been determined by locating the peak centers using the relatively narrow window, processing may be returned for iterations on finding a Gaussian fit, for a more accurate fit. Since the spacing is now known, a window which is about half the peak spacing can be used to do the Gaussian integration to fit the Gaussian curves for the peaks.

Grouping the Peaks

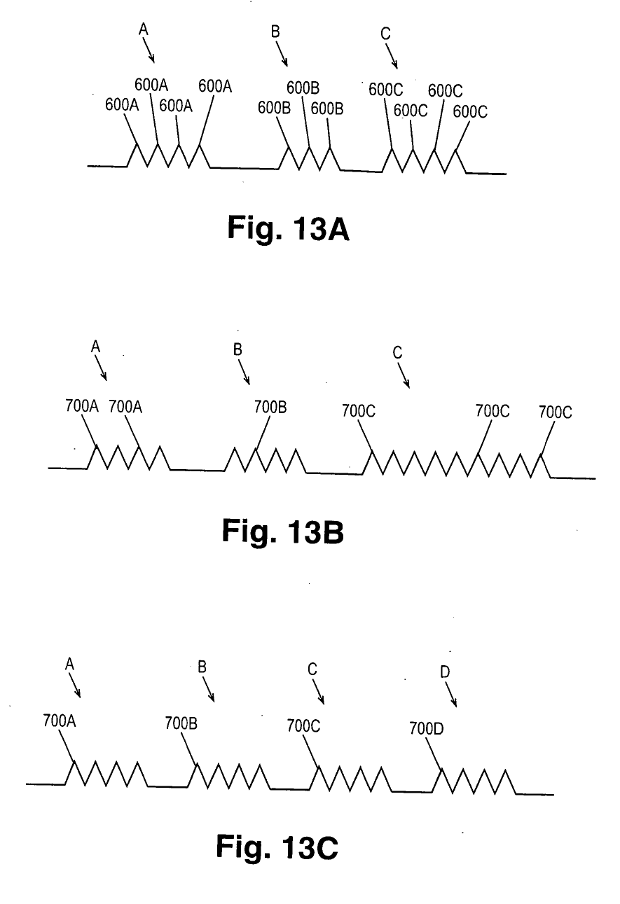

After the peak centers, peak spacing and peak widths have been established, according to the above methods, the system further processes the data to establish peak grouping. Peak grouping relates to the features as they are arranged in microarrays, for example. In certain situations, a consistent repeating pattern of peak grouping may be observable in the data, while one or more such groups may deviate slightly from the established pattern. For example, FIG. 13A shows a repeating pattern of four peaks consistently spaced, with each group being relatively consistently spaced by a larger distance than the distance between peaks within the group. The peaks 600A and 600C in groups A and C fit this pattern well. However, group B shows only three peaks 600B and is spaced somewhat further from group A than it is from group C. The program, in this type of situation, recognizes the established pattern of groups, the group spacing and the peak spacing. By comparing the average group spacings over the entire data set, and comparing this with the group spacing and number of peaks in group B as compared with the other groups, group B will be arranged to account for the missing peak (first peak in the group in this instance) to maintain proper alignment of this group with the other microarrays.

Another anomalous situation that may occur is like that shown in FIG. 13B. In this situation, there is again a regular pattern of four peaks established in most groups, such as shown by groups A and B, and each group is regularly spaced. However, group C shows nine peaks 700C. In this case, the program compares the peak spacings in group C with the other groups' peak spacings, and considers whether group C can be broken down into two groups while maintaining the established peak spacing and group spacing. In this example, the fifth identified “peak” was likely caused by noise of a form discussed above. By eliminating the fifth peak, the system determines that group C can actually be represented as two groups (i.e., groups C and D, as shown in FIG. 13C) and that this representation fits the established number of peaks per group as well as the established group spacing. Hence, the system filters out the fifth peak 700C and represents what was group C as new groups C and D, each containing four consistently spaced peaks and each having consistent group spacing.

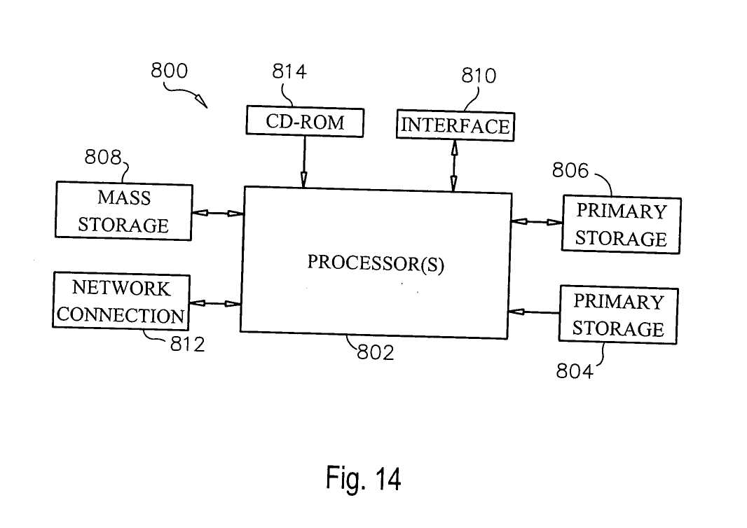

FIG. 14 illustrates a typical computer system in accordance with an embodiment of the present invention. The computer system 800 includes any number of processors 802 (also referred to as central processing units, or CPUs) that are coupled to storage devices including primary storage 806 (typically a random access memory, or RAM), primary storage 804 (typically a read only memory, or ROM). As is well known in the art, primary storage 804 acts to transfer data and instructions uni-directionally to the CPU and primary storage 806 is used typically to transfer data and instructions in a bi-directional manner Both of these primary storage devices may include any suitable computer-readable media such as those described above. A mass storage device 808 is also coupled bi-directionally to CPU 802 and provides additional data storage capacity and may include any of the computer-readable media described above. Mass storage device 808 may be used to store programs, data and the like and is typically a secondary storage medium such as a hard disk that is slower than primary storage. It will be appreciated that the information retained within the mass storage device 808, may, in appropriate cases, be incorporated in standard fashion as part of primary storage 806 as virtual memory. A specific mass storage device such as a CD-ROM 814 may also pass data uni-directionally to the CPU.

CPU 802 is also coupled to an interface 810 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 802 optionally may be coupled to a computer or telecommunications network using a network connection as shown generally at 812. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for clustering vectors may be stored on mass storage device 808 or 814 and executed on CPU 808 in conjunction with primary memory 806.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of automatically separating multiple microarray images provided as a single combined image of the multiple microarray images, said method comprising the steps of:
providing at least one image containing multiple microarray images thereon, each microarray image comprising a plurality of features;
automatically locating the features in the microarray images;
automatically determining the boundaries of each microarray image based on the locations of the features; and
automatically cropping the image containing multiple microarray images to form a group of single images, each containing only one microarray image cropped from the image containing multiple microarray images.

2. The method of claim 1, wherein said cropping is performed at locations measured from the determined image boundaries and offset by predetermined boundary parameters.

3. The method of claim 2, further comprising user input of said predetermined boundary parameters.

4. The method of claim 2, wherein said predetermined boundary parameters are default parameters that are automatically applied during said cropping.

5. The method of claim 1, wherein said automatically locating the features is performed using a projection-based algorithm.

6. The method of claim 5, wherein the multiple microarray images are displayed in a two-dimensional array, and wherein said automatically locating and automatically determining comprise:
projecting the two dimensional array in a first of the two dimensions to form a one dimensional dataset representative of the values in the first dimension;
peak picking the one dimensional dataset and determining which picked peaks to retain for further processing, based on predetermined peak height and peak width thresholds;
estimating spacing between the features based on a statistical determination of a most frequent distance between centers of retained peaks which are adjacent one another;
projecting the two dimensional array in the second of the two dimensions to form a one dimensional dataset representative of the values in the second dimension;
peak picking the one dimensional dataset representative of the values in the second dimension, and determining which picked peaks to retain for further processing, based on predetermined peak height and peak width thresholds;
estimating spacing between the features based on a statistical determination of a most frequent distance between centers of retained peaks which are adjacent one another; and
generating coordinates for the features on the array, relative to X and Y axes referring to the first and second dimensions, based on the picked peaks and peak spacing.

7. The method of claim 1, further comprising inputting, by a user, cropping parameters according to which to automatically crop the images.

8. The method of claim 1, further comprising automatically naming the single images.

9. The method of claim 1, further comprising automatically storing the single images as separate files.

10. The method of claim 9, further comprising inputting, by a user, a storage location in which said single images are automatically stored.

11. The method of claim 8, further comprising automatically storing the named, single images as separate files.

12. The method of claim 11, further comprising inputting, by a user, a storage location in which said named, single images are automatically stored.

13. The method of claim 8, further comprising inputting, by a user, names to be applied to said single images during said automatically naming said single images.

14. The method of claim 1, wherein said providing at least one image containing multiple microarray images comprises providing a plurality of images each containing multiple microarray images, and wherein said automatically locating the features, automatically determining the boundaries, and automatically cropping are performed on each of the images containing multiple microarray images, in batch mode.

15. A method comprising forwarding a result obtained from the method of claim 1 to a remote location.

16. A method comprising transmitting data representing a result obtained from the method of claim 1 to a remote location.

17. A method comprising receiving a result obtained from a method of claim 1 from a remote location.

18. A method of evaluating separation locations of multiple microarray images in a single combined image of the multiple microarray images, and separating the multiple microarray images along the separation locations, wherein the images are represented in a two-dimensional array, said method comprising the steps of:
projecting the two dimensional array in a first dimension to form a one-dimensional dataset representative of values of features located in the microarray images in the first dimension;
projecting the two dimensional array in a second dimension to form a one-dimensional dataset representative of values of features located in the microarray images in the second dimension;
evaluating the one-dimensional datasets for spacing patterns in the first and second one-dimensional datasets indicative of separations between the microarray images; and
separating the microarray images based on the locations of separations identified by said evaluating.

19. A system for automatically cropping microarray images from an image containing multiple microarray images, said system comprising:
means for receiving at least one image containing multiple microarray images thereon, each microarray image comprising a plurality of features;
means for automatically locating the features in the microarray images;
means for automatically determining the boundaries of each microarray image based on the locations of the features; and
means for automatically cropping the image containing multiple microarray images to form a group of single images, each containing only one microarray image cropped from the image containing multiple microarray images.

20. The system of claim 19, wherein said cropping is performed at locations measured from the determined image boundaries and offset by predetermined boundary parameters.

21. The system of claim 20, further comprising a user interface including means for user input of said predetermined boundary parameters.

22. The system of claim 20, wherein said predetermined boundary parameters are default parameters that are automatically applied during said cropping.

23. The system of claim 19, wherein said means for automatically locating the features comprises means for applying a projection-based algorithm.

24. The system of claim 19, wherein the multiple microarray images are displayed in a two-dimensional array, and wherein said means for automatically locating and means for automatically determining comprise:
means for projecting the two dimensional array in a first of the two dimensions to form a one dimensional dataset representative of the values in the first dimension;
means for peak picking the one dimensional dataset and determining which picked peaks to retain for further processing, based on predetermined peak height and peak width thresholds;
means for estimating spacing between the features based on a statistical determination of a most frequent distance between centers of retained peaks which are adjacent one another;
means for projecting the two dimensional array in the second of the two dimensions to form a one dimensional dataset representative of the values in the second dimension;
means for peak picking the one dimensional dataset representative of the values in the second dimension, and determining which picked peaks to retain for further processing, based on predetermined peak height and peak width thresholds;
means for estimating spacing between the features based on a statistical determination of a most frequent distance between centers of retained peaks which are adjacent one another; and
means for generating coordinates for the features on the array, relative to X and Y axes referring to the first and second dimensions, based on the picked peaks and peak spacing.

25. The system of claim 19, further comprising a user interface including means for user input of cropping parameters according to which to automatically crop the images.

26. The system of claim 19, further comprising means for automatically naming the single images.

27. The system of claim 19, further comprising means for automatically storing the single images as separate files.

28. The system of claim 27, further comprising a user interface including means for user input of a storage location in which said single images are automatically stored.

29. The system of claim 26, further comprising means for automatically storing the named, single images as separate files.

30. The system of claim 29, further comprising a user interface including means for user input of a storage location in which said named, single images are automatically stored.

31. The system of claim 26, further comprising a user interface including means for user input of names to be applied to said single images during said automatically naming said single images.

32. The system of claim 19, wherein said means for receiving is capable of receiving a plurality of images each containing multiple microarray images, and wherein said system comprises means for automatically batch processing said plurality of images.

33. A computer readable medium carrying one or more sequences of instructions for automatically separating multiple microarray images provided as a single combined image of the multiple microarray images, wherein execution of one or more sequences of instructions by one or more processors causes the one or more processors to perform the steps of:
automatically locating the features in the microarray images;
automatically determining the boundaries of each microarray image based on the locations of the features; and
automatically cropping the image containing multiple microarray images to form a group of single images, each containing only one microarray image cropped from the image containing multiple microarray images.

34. The computer readable medium of claim 33, wherein the multiple microarray images are displayed in a two-dimensional array, and wherein said automatically locating the features and automatically determining the boundaries comprise the steps of:
projecting the two dimensional array in a first of the two dimensions to form a one dimensional dataset representative of the values in the first dimension;
peak picking the one dimensional dataset and determining which picked peaks to retain for further processing, based on predetermined peak height and peak width thresholds;
estimating spacing between the features based on a statistical determination of a most frequent distance between centers of retained peaks which are adjacent one another;
projecting the two dimensional array in the second of the two dimensions to form a one dimensional dataset representative of the values in the second dimension;
peak picking the one dimensional dataset representative of the values in the second dimension, and determining which picked peaks to retain for further processing, based on predetermined peak height and peak width thresholds;
estimating spacing between the features based on a statistical determination of a most frequent distance between centers of retained peaks which are adjacent one another; and
generating coordinates for the features on the array, relative to X and Y axes referring to the first and second dimensions, based on the picked peaks and peak spacing.
